(19) United States
(12) Patent Application Publication
(10) Pub. No.: US 2024/0058149 A1
(43) Pub. Date: Feb. 22, 2024

(54) Sleep Aid for Arthritic Pain and Stiffness in Lower Back

(71) Applicant: Vanderbilt Brabson, III, Corryton, TN (US)

(72) Inventor: Vanderbilt Brabson, III, Corryton, TN (US)

(21) Appl. No.: 18/082,231

(22) Filed: Dec. 15, 2022

Related U.S. Application Data
(60) Provisional application No. 63/399,476, filed on Aug. 19, 2022.

(51) Int. Cl. A61F 5/01

(52) U.S. Cl. CPC A61F 5/0102; A61F 2005/0146

(57) ABSTRACT

A plurality of round members, such as balls, that are coupled together in an array by a connecting agent such as one or more nylon cords that allow the balls to move and work in concert and uniformity with the lower back muscles and movement of a sleeping user lying upon the device. With such use the connected and movable balls provide continuous acupressure throughout the night as the user sleeps, resulting in continuous relief and mitigation of arthritic pain and stiffness in one's lower back throughout the night. Thus, pain and stiffness which would otherwise greatly disturb one's attempt to sleep through the night is mitigated, if not outright relieved.

### CROSS-REFERENCE TO RELATED APPLICATIONS

This application claims the benefit of U.S. Provisional Patent Application Ser. No. 63/399,476, filed Aug. 19, 2022, the entire content of which is incorporated herein by reference.

### FIELD OF INVENTION

The present general inventive concept relates to therapeutic devices, and, more particularly, to a device to alleviate lower back discomfort.

### BACKGROUND

Back pain, particularly lower back pain, is not only problematic during waking hours, it can make it nearly impossible to have quality sleep. Therefore, it would be desirable to have a device on which a patient could lie that would provide relief and/or therapy for such discomfort.

### SUMMARY

A plurality of round members, such as balls, that are coupled together in an array by a connecting agent such as one or more nylon cords that allow the balls to move and work in concert and uniformity with the lower back muscles and movement of a sleeping user lying upon the device. With such use the connected and movable balls provide continuous acupressure throughout the night as the user sleeps, resulting in continuous relief and mitigation of arthritic pain and stiffness in one's lower back throughout the night. Thus, pain and stiffness which would otherwise greatly disturb one's attempt to sleep through the night is mitigated, if not outright relieved.

In various example embodiments the balls are threaded together such that they may bunch up and over one another with some movements, but be easily spread out to a generally even plane by moving a user's hand or other the like over the balls to smooth them out. In various example embodiments each of the round members are configured to movable relative to any adjacent one of the round members. The interconnected balls or generally round members may be enveloped in a sleeve or cover that may be easily removable, and which may be washable.

In some example embodiments according to the present general inventive concept, a therapeutic device for sleeping comprises at least one planar assembly of round members connected by cords threaded through said round members, said cords being threaded and interlaced into a grid-like pattern, the round members being arranged to be movable and to provide acupressure to a sleeper lying thereon.

Some embodiments further comprise a removable cover configured to hold the array therein.

In some embodiments, the removable cover is fabricated from a cloth material.

In some embodiments, the round members are interconnected by a nylon cord passing through the round members.

In some embodiments, each of the round members is configured to be movable relative to any adjacent one of the round members.

In some embodiments, the planar assembly of round members includes an array of round members that is dimensioned three round members wide, fourteen round members long, and one round member thick.

Some embodiments comprise two planar assembly of round members stacked one planar assembly atop the other planar assembly.

Some embodiments comprise three planar assembly of round members stacked in layers.

In some embodiments, each of the round members is fabricated from a plastic material.

In some embodiments, each of the round members is fabricated from a plastic material with a Shore D hardness from 55 to 100.

In some embodiments, each of the round members is fabricated from a plastic material with a Shore D hardness from 65 to 80.

In some example embodiments according to the present general inventive concept, a therapeutic device for sleeping comprises a plurality of connected and movable round members coupled together in an array by a connecting agent that allows the round members to move and work in concert and uniformity with the lower back muscles and movement of a sleeping user lying upon the therapeutic device, whereby the connected and movable round members provide continuous acupressure throughout the night as the user sleeps, resulting in continuous relief and mitigation of arthritic pain and stiffness in one's lower back throughout the night.

Some embodiments further include a removable cover configured to hold the array therein.

In some embodiments, the connecting agent comprises a nylon cord.

In some embodiments, each of the round members is configured to movable relative to any adjacent one of the round members.

Various example embodiments of the present general inventive concept may provide a plurality of covers in different colors, materials, thicknesses, and so on, or any combination thereof. The ball members may be made of a variety and range of materials. Various example embodiments may provide hollow plastic balls that are lightweight but structurally sufficient to not be collapsed or dented by the user's weight. Such ball members may be similar to light practice golf balls. Various example embodiments may provide enough slack to the tying members to allow the balls to move apart from one another a predetermined distance to allow for greater malleability in overall shape. Various example embodiments may provide an elastic tying member so as to allow the ball members to be able to at least somewhat separate from each other to provide comforting contact during movement or different positions.

### BRIEF DESCRIPTION OF THE FIGURES

The following example embodiments are representative of example techniques and structures designed to carry out the objects of the present general inventive concept, but the present general inventive concept is not limited to these example embodiments. In the accompanying drawings, illustrations, and photographs, the sizes and relative sizes, shapes, and qualities of lines, entities, and regions may be exaggerated for clarity. A wide variety of additional embodiments will be more readily understood and appreciated through the following detailed description of the example embodiments.

The above-mentioned and additional features of the invention will become more clearly understood from the following detailed description of the invention read together with the drawings in which:

FIG. 1 is a perspective view of a sleep aid with an array of round members, according to one example embodiment of the present general inventive concept;

FIG. 2 is a second perspective view of the example embodiment sleep aid with an array of round members shown in FIG. 1, showing a partially transparent view of the array of round members positioned inside a cover;

FIG. 3 is a top view of the example embodiment sleep aid with an array of round members shown in FIGS. 1 and 2, without the cover;

FIG. 4 is a second top view of the example embodiment sleep aid with an array of round members shown in FIGS. 1-3, showing a partially transparent view of the array of round members positioned inside a cover;

FIG. 5 is a section view of the example embodiment shown in FIGS. 1-4, illustrating the connected relationship between adjacent round members within the array of round members;

FIG. 6 is a second section view of the example embodiment shown in FIG. 5;

FIG. 7 is a top view of a second example embodiment of the present general inventive concept;

FIG. 8 is a section view of the example embodiment shown in FIG. 7;

FIG. 9A is an exploded perspective view of another example embodiment of the present general inventive concept, incorporating multiple layers of arrayed round members;

FIG. 9B is a top view of the example embodiment shown in FIG. 9A; and

FIG. 10 is a section view of the example embodiment shown in FIGS. 9A and 9B.

### DETAILED DESCRIPTION

Reference will now be made to the example embodiments of the present general inventive concept, examples of which are illustrated in the accompanying drawings and illustrations. The example embodiments are described herein in order to explain the present general inventive concept by referring to the figures.

The following detailed description is provided to assist the reader in gaining a comprehensive understanding of the structures and fabrication techniques described herein. Accordingly, various changes, modification, and equivalents of the structures and fabrication techniques described herein will be suggested to those of ordinary skill in the art. Progressions of fabrication operations described are merely examples, however, and the sequence of operations is not limited to that set forth herein and may be changed as is known in the art, with the exception of operations necessarily occurring in a certain order. Also, description of well-known functions and constructions may be simplified and/or omitted for increased clarity and conciseness.

Note that spatially relative terms, such as “up,” “down,” “right,” “left,” “beneath,” “below,” “lower,” “above,” “upper” and the like, may be used herein for ease of description to describe one element or feature's relationship to another element(s) or feature(s) as illustrated in the figures. Spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

According to various example embodiments of the present general inventive concept, a sleep aid is provided that relieves arthritic pain and stiffness in the lower back during the night as the user sleeps.

Various example embodiments of the sleep aid include a plurality of round members, such as balls, that are coupled together in an array by a connecting agent such as one or more nylon cords that allow the balls to move and work in concert and uniformity with the lower back muscles and movement of a sleeping user lying upon the device. With such use the connected and movable balls provide continuous acupressure throughout the night as the user sleeps, resulting in continuous relief and mitigation of arthritic pain and stiffness in the user's lower back throughout the night. Thus, pain and stiffness which would otherwise greatly disturb the user's attempt to sleep through the night is mitigated, if not altogether eliminated or totally relieved.

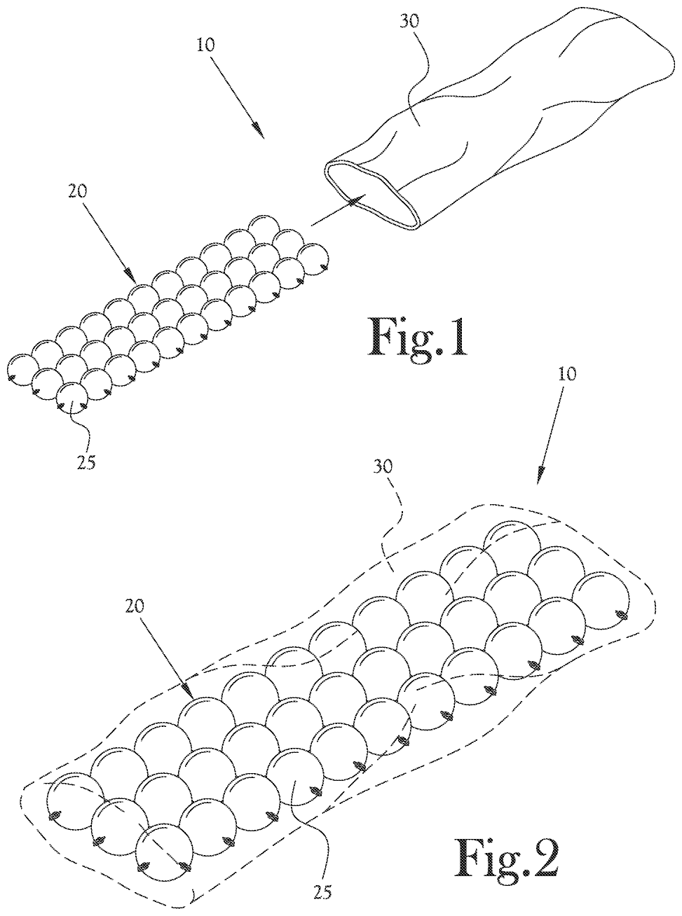

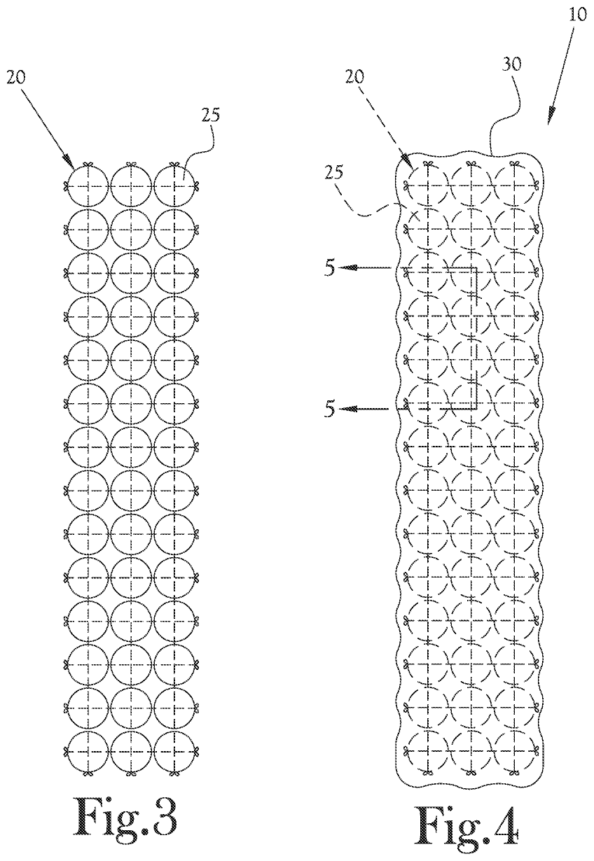

Turning to the Figures, FIG. 1 is a perspective view of a therapeutic sleep aid 10 with an array of round members, according to one example embodiment of the present general inventive concept, illustrating in a general way a planar array 20 of round members 25 connected by cords threaded through the round members 25. FIG. 1 shows the planar array 20 situated outside a removable cover 30, and FIG. 2, in a partially transparent perspective view, shows the planar array 20 of round members 25 connected by cords threaded through the round members 25 inside the cover 30. FIG. 3 is a top view of the example embodiment therapeutic sleep aid 10 with the planar array 20 of round members 25, without the cover. FIG. 4 is a second top view of the example embodiment sleep aid with an array of round members shown in FIGS. 1-3, showing a partially transparent view of the array 20 of round members 25 positioned inside the cover 30. FIG. 3 illustrates the array 20 in itself, without a cover, and FIG. 4 illustrates the array 20 inside an example cover 30.

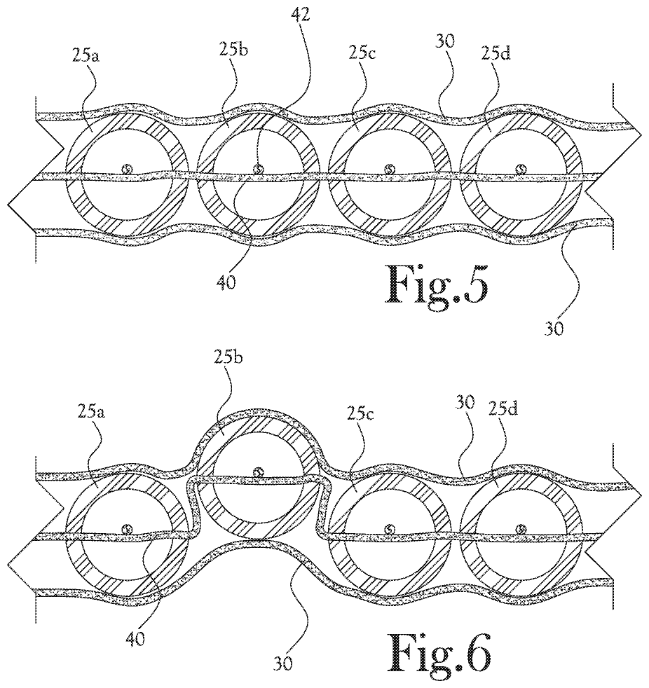

As illustrated in FIGS. 1-4, an array 20 of interconnected round members or balls 25 are configured to stay connected but to be movable to fit the contours of a user's lower back, but at the same time form a discontinuous surface which provides therapeutic acupressure on the lower back. As shown in the section view illustrated in FIG. 5, in various example embodiments the round members are threaded together by a nylon cord 40 such that they may bunch up and over one another with some movements, but be easily spread out to a generally even plane by moving a user's hand or other the like over the round members to smooth them out. In various example embodiments each of the round members are configured to be movable relative to any adjacent one of the round members.

The interconnected balls or generally round members may be enveloped in a sleeve or cover 30 that may be easily removable, and which may be washable. Various example embodiments of the present general inventive concept may provide a plurality of covers in different colors, materials, thicknesses, and so on, or any combination thereof.

FIG. 5 is a section view of the example embodiment shown in FIGS. 1-4, illustrating the connected relationship between adjacent round members within the array of round members; the shown example round members 25a-d are threaded together by a nylon cord 40; throughout the planar array, cords are threaded and interlaced into a grid-like pattern, helping to produce and maintain the grid-like pattern of round members 25 seen in FIGS. 3 and 4. For example, within the round member 25b, a first nylon cord 40 and a second nylon cord 42 cross at substantially right angles. FIG. 6 is a second section view of the example embodiment shown in FIG. 5; as shown in FIG. 6, the nylon cord 40 (or other elastic connecting member, in other embodiments) provides enough slack to permit the round member 25b to move with respect to its adjacent round members 25a and 25c, with the maximum permitted movement by any one round member predetermined by the material properties of the nylon cord 40.

The round members or balls may be made of a host of materials. Various example embodiments may provide hollow plastic balls that are lightweight but structurally sufficient to not be collapsed or dented by the user's weight. Such ball members may be similar to light practice golf balls. While the example embodiments illustrated in FIGS. 1-4 show the balls as being connected in close contact with one another, various example embodiments may provide enough slack to the tying members to allow the balls to move apart from one another a predetermined distance to allow for greater malleability in overall shape. Various example embodiments may provide an elastic tying member so as to allow the ball members to be able to at least somewhat separate from each other to provide comforting contact during movement or different positions.

In some embodiments, each of the round members is fabricated from a plastic material. In some embodiments, each of the round members is fabricated from a plastic material with a Shore D hardness from 55 to 100. In some embodiments, each of the round members is fabricated from a plastic material with a Shore D hardness from 65 to 80. Other workable materials will be apparent to those of skill in the art and are contemplated by the present general inventive concept and the present disclosure.

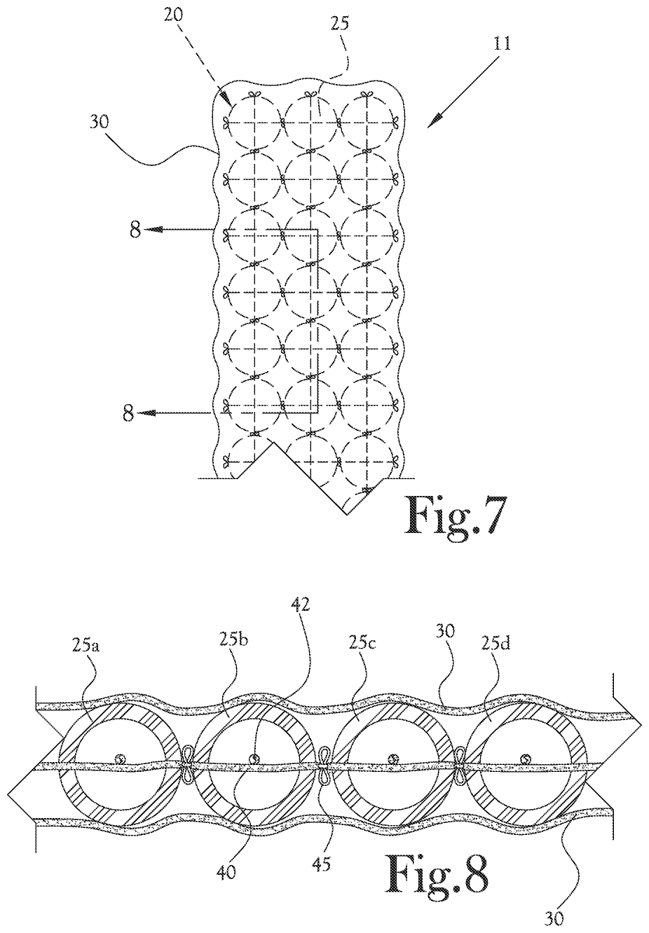

FIG. 7 is a top view of a second example embodiment 11 of the present general inventive concept; and FIG. 8 is a section view of the example embodiment 11 shown in FIG. 7. FIGS. 7 and 8 show a second example embodiment 11 in which the planar array 20 of round members 25 are connected by cords 40 with knots 45 in between each adjacent round member 25. Other arrangements will be apparent to those of skill in the art and are contemplated by the present general inventive concept and the present disclosure.

While the Figures and drawings show a certain number of ball members arranged in the array, such as 3×14, it is understood that various example embodiments may provide different numbers of ball members without departing from the scope of the present general inventive concept. Various example embodiments have a length sufficient to cover the entire lower back of the user from the user's left to right side, and a width sufficient to cover the lower back from “top” to “bottom.” Further, while the illustrated example embodiments show generally straight rows and columns of the ball member array, differently arranged configurations, such as diagonally aligned ball members, may be employed without departing from the scope of the present general inventive concept.

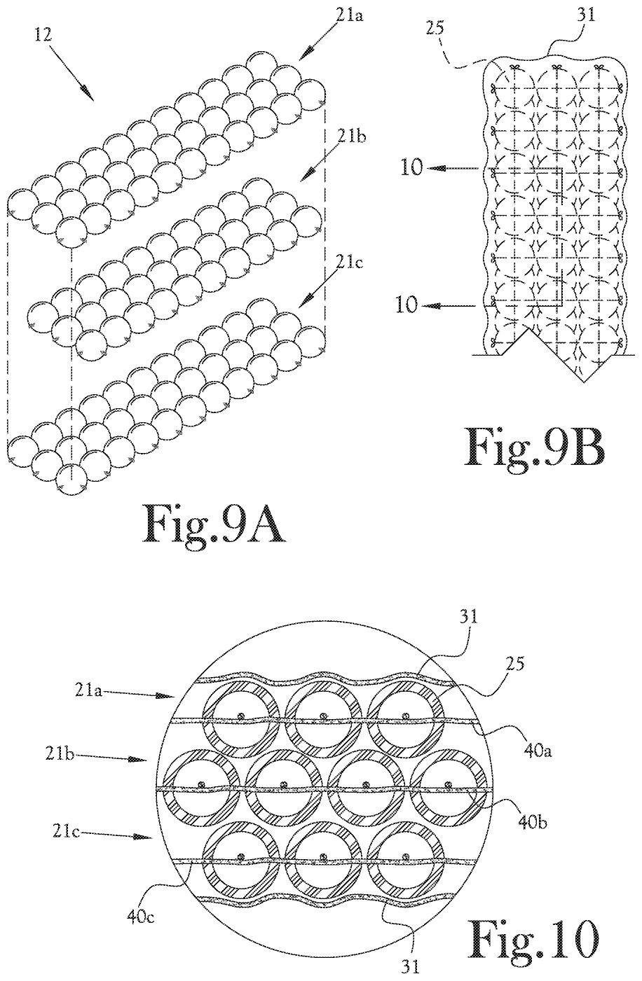

By way of further example, FIG. 9A is an exploded perspective view of another example embodiment of the present general inventive concept, incorporating multiple layers of arrayed round members. FIG. 9B is a top view of the same example embodiment shown in FIG. 9A, and FIG. 10 is a section view of the example embodiment shown in FIGS. 9A and 9B. As shown in those Figures, the illustrated example embodiment assembly 12 includes three layers of arrayed round members 21a, 21b, 21c, positioned one layer atop another. As shown in the section view in FIG. 10, the round members 25 are generally staggered within the cover 31, so that the round members of the separate layers 21a, 21b, 21c fit together in a close packing structural arrangement, such as a hexagonal close packing arrangement or a face-centered cubic packing arrangement. Other close packing arrangements will be apparent to those of skill in the art and are contemplated by the present general inventive concept and the present disclosure.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

It is noted that the simplified diagrams and drawings included in the present application do not illustrate all the various connections and assemblies of the various components, however, those skilled in the art will understand how to implement such connections and assemblies, based on the illustrated components, figures, and descriptions provided herein, using sound engineering judgment. Numerous variations, modification, and additional embodiments are possible, and, accordingly, all such variations, modifications, and embodiments are to be regarded as being within the spirit and scope of the present general inventive concept.

While the present general inventive concept has been illustrated by description of several example embodiments, and while the illustrative embodiments have been described in detail, it is not the intention of the applicant to restrict or in any way limit the scope of the general inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings. Additional modifications will readily appear to those skilled in the art. Accordingly, departures may be made from such details without departing from the spirit or scope of applicant's general inventive concept.

1. A therapeutic device for sleeping, comprising:
at least one planar assembly of round members connected by cords threaded through said round members, said cords being threaded and interlaced into a grid-like pattern, said cords including longitudinal cords and lateral cords, both longitudinal cords and lateral cords being threaded through said round members so that each round member is threaded by one longitudinal cord and one lateral cord, each round member being connected by a shared cord to at least one longitudinally adjacent round member and connected by a shared cord to at least one laterally adjacent round member,
the round members being arranged to be movable and to provide acupressure to a sleeper lying thereon.

2. The therapeutic device of claim 1, further comprising a removable cover configured to hold the array therein.

3. The therapeutic device of claim 2, wherein said removable cover is fabricated from a cloth material.

4. The therapeutic device of claim 1, wherein the cords threaded through said round members include nylon cords.

5. The therapeutic device of claim 1, wherein each of the round members is configured to movable relative to any adjacent one of the round members.

6. The therapeutic device of claim 1, wherein the planar assembly of round members includes an array of round members that is dimensioned three round members wide, fourteen round members long, and one round member thick.

7. The therapeutic device of claim 1 comprising two planar assembly of round members stacked one planar assembly atop the other planar assembly.

8. The therapeutic device of claim 1 comprising three planar assembly of round members stacked in layers.

9. The therapeutic device of claim 1, wherein each of the round members is fabricated from a plastic material.

10. The therapeutic device of claim 9, wherein each of the round members is fabricated from a plastic material with a Shore D hardness from 55 to 100.

11. The therapeutic device of claim 10, wherein each of the round members is fabricated from a plastic material with a Shore D hardness from 65 to 80.

12. A therapeutic device for sleeping, comprising:
a plurality of connected and movable round members coupled together in an array by a connecting agent that allows the round members to move and work in concert and uniformity with the lower back muscles and movement of a sleeping user lying upon the therapeutic device, said connecting agent including longitudinal connectors and lateral connectors, both longitudinal connectors and lateral connectors being threaded through said round members so that each round member is threaded by one longitudinal connector and one lateral connector, each round member being connected by a shared connector to at least one longitudinally adjacent round member and connected by a shared connector to at least one laterally adjacent round member,
whereby the connected and movable round members provide continuous acupressure throughout the night as the user sleeps, resulting in continuous relief and mitigation of arthritic pain and stiffness in one's lower back throughout the night.

13. The therapeutic device of claim 12, further comprising a removable cover configured to hold the array therein.

14. The therapeutic device of claim 12, wherein the connecting agent comprises a nylon cord.

15. The therapeutic device of claim 12, wherein each of the round members is configured to movable relative to any adjacent one of the round members
